(19) United States
(12) Patent Application Publication
(10) Pub. No.: US 2026/0188508 A1
(43) Pub. Date: Jul. 2, 2026

(54) ASSESSING HEALTH OF A HEART VALVE

(71) Applicants: Universitetet I Tromsø - Norges Arktiske Universitet, Tromsø (NO); Medsensio AS, Tromsø (NO); Universitetet I Oslo, Oslo (NO)

(72) Inventors: LARS AILO ASLAKSEN BONGO, Tromsø (NO); PER NIKLAS BENZLER WAALER, Tromsø (NO); HASSE MELBYE, Tromsø (NO); MARKUS KREUTZER JOHNSEN, Tromsø (NO); JOHAN FREDRIK EGGEN RAVN, Tromsø (NO); HENRIK SCHIRMER, Oslo (NO); JUAN CARLOS AVILES SOLIS, Tromsø (NO); TOM DØNNEM, Tromsø (NO); STIAN ANDERSEN, Tromsø (NO); Anne Herefoss DAVIDSEN, Tromsø (NO)

(21) Appl. No.: 19/131,342

(22) PCT Filed: Nov. 22, 2023
(86) PCT No.: PCT/EP2023/082721

§ 371 Date: May 20, 2025

(30) Foreign Application Priority Data
Nov. 23, 2022 (GB) 2217512.9

(51) Int. Cl. G16H 50/30; A61B 5/00; A61B 5/02; A61B 7/04

(52) U.S. Cl. CPC G16H 50/30; A61B 5/02028; A61B 5/7267; A61B 7/04

(57) ABSTRACT

Computer software, a computer system (212) and a corresponding method for assessing health of a heart valve of a heart of a subject (202) are disclosed. A plurality of murmur-grade values are received, wherein each murmur-grade value is associated with a different respective auscultation position (102, 104, 106, 108). The plurality of murmur-grade values are input to a first trained software model (304), implemented by the computer software. The first trained software model (304) is operated to generate a value indicative of health of the heart valve. The value indicative of health of the heart valve is output from the first trained software model (304).

### BACKGROUND OF THE INVENTION

This invention relates to methods, software and systems for assessing valvular health of a heart.

The hearts of human and animal subjects can become more or less healthy over time, depending on factors such as lifestyle, diet, exercise, etc. It is desirable to monitor heart health in order to determine if any lifestyle or medical interventions are indicated, or may become necessary in future. One aspect of heart function that is beneficial to assess is the health of the valves, such as the aortic valve.

The presence and degree of any audible murmur, due to blood flow turbulence, can be determined by a physician using a stethoscope to listen to the heart. This can provide an indication of the valvular health of the heart. The physician may assign a numerical murmur grade, as an integer between one and six for a murmur, to indicate the degree of any detected murmur. An identification of a particular valve involved in the murmur can be made based upon the timing of the murmur within the cardiac cycle and upon where the murmur is loudest—i.e. at which of four standard auscultation positions, located adjacent respective valves of the four heart valves. The murmur grade may be used to determine whether further investigation is warranted, such as a referral for an echocardiogram.

An echocardiogram uses Doppler ultrasound to provide far greater insight into the health of the heart, such as calculating a time-averaged pressure gradient across one or more valves of the heart. Such information can be used to facilitate a more complete assessment of the health of a heart valve, including potentially diagnosing a valvular heart disease (VHD) such as aortic stenosis (AS).

Although the stethoscope can be a cost-effective tool for assessing cardiovascular health, e.g. to determine if a referral for an echocardiogram is appropriate, significant time and skill is required to perform accurate cardiac auscultation and interpret the findings. It also requires the clinician to have good hearing. While an echocardiogram is accurate, it is expensive. It also requires highly trained personnel to analyze its results. Echocardiograms cannot therefore replace the stethoscope as a front-line tool for assessing valvular heart health, especially in low-income countries where availability to echocardiography is limited.

It is therefore desirable to have an alternative approach to assessing valvular heart health that can mitigate at least some of these limitations of conventional approaches.

### SUMMARY OF THE INVENTION

From a first aspect, the invention provides a computer-implemented method for assessing health of a heart valve of a heart of a subject, the method comprising:
receiving a plurality of murmur-grade values, wherein each murmur-grade value is associated with a different respective auscultation position;
inputting the plurality of murmur-grade values to a first trained software model;
operating the first trained software model to generate a value indicative of health of the heart valve; and
outputting the value indicative of health of the heart valve from the first trained software model.

From a second aspect, the invention provides computer software for assessing health of a heart valve of a heart of a subject, wherein the software comprises instructions which, when executed on a processing system, cause the processing system to:
receive a plurality of murmur-grade values, wherein each murmur-grade value is associated with a different respective auscultation position;
input the plurality of murmur-grade values to a first trained software model implemented by the computer software;
operate the first trained software model to generate a value indicative of health of the heart valve; and
output the value indicative of health of the heart valve from the first trained software model.

From a third aspect, the invention provides a computer system for assessing the health of a heart valve of a heart of a subject, wherein the computer system comprises a processing system and a memory storing computer software as disclosed herein, for execution by the processor.

Thus it will be seen that, in accordance with embodiments of the invention, valuable insight into the health of a heart valve can be obtained by using a trained software model to analyse murmur-grade values associated with multiple different auscultation positions. This can reduce the dependence on human skill, and may lead to cheaper and/or more accurate assessments. Murmur-grade values associated with different auscultation positions may be more easily determined than other data, such as Doppler ultrasound measurements, commonly used to assess the valvular health of the heart, that can only be obtained by in-depth examination using expensive equipment. Furthermore, inputting murmur-grade values from a plurality of different auscultation positions into the trained software model, rather than just a single auscultation position, to assess the health of a particular heart valve, allows the murmur-grade values at different positions to be considered in the analysis, rather than analysing a murmur-grade value associated with a single auscultation position in isolation.

The subject may be an animal, but in some embodiments the subject is a human. The valve may be any valve, but in some embodiments it is an aortic valve.

The value indicative of health of the heart valve may be used for any of various purposes. In a set of embodiments, the value may be predictive of (e.g. correlate with a likelihood of) having a valvular heart disease (VHD). It may be used to determine whether to refer the subject for further investigation, such as an echocardiogram, to determine whether or not the subject has VHD. However this is not essential, and in other embodiments it may be used to monitor heart health. The value indicative of valve health may aid in the identification of any of various conditions, but in a set of embodiments, the value is indicative of the presence of stenosis of the valve, e.g. of aortic stenosis (AS). The method may comprise comparing the value indicative of valve health with one or more threshold values, and may comprise assigning one of a predetermined set of different health categories in dependence on the comparison. In a set of embodiments, the value indicative of health of the heart valve is compared to a threshold to determine whether AS is present. However comparing the value with a standard value is not essential, and the value may instead be stored and/or compared with one or more values determined previously by the same method, e.g. to monitor changes in heart health over time.

In a set of embodiments, the first trained software model may be arranged to output a value indicative of health of the heart valve that correlates (positively or negatively) with pressure gradient across the valve. It may scale linearly with the pressure gradient. The value indicative of health of the heart valve could be a measure of the peak velocity of blood flow through the aortic valve, but in a set of embodiments it is a pressure gradient. The pressure gradient may be pressure difference, or it may be a measure of the change in pressure over a distance. The pressure gradient could be an instantaneous or peak value, but in some embodiments it is a time-averaged mean pressure gradient (e.g. averaged over one or more cardiac cycles). The valve may be the aortic valve and the pressure gradient may be an aortic valve pressure gradient (e.g. a mean AVPG). Such a value, output by the model, will be referred to herein as an AVPG value. A low mean AVPG may indicate that the aortic valve of the heart is functioning effectively. Conversely, a high mean AVPG may indicate that narrowing of the aortic valve has occurred, resulting in aortic stenosis (AS). Accordingly, the subject may be indicated for further investigation (e.g. for potential diagnosing of AS) if the generated AVPG value exceeds a predetermined threshold.

Each murmur-grade value may be associated with a severity of murmur present at the respective auscultation position. It may be a value on the Levine scale (optionally modified to include fractional values rather than integer values only as is typical). Each murmur-grade value may have a value between a lower bound (e.g. zero or one) and an upper bound (e.g. six). Each murmur-grade value may be received as an integer value, which may be a single-digit integer. However, in a set of embodiments, the first trained software model is arranged to receive integer and non-integer murmur-grade values, e.g. with each received murmur-grade value having an integer component and one or more decimals digits (which may be zero on occasions, but which are non-zero in other instances). Advantageously, using murmur grades that include fractional components, rather than a binary variable or single-digit integer classification, can allow the model to make use of more detailed information about the murmur. Furthermore, supporting fractional numerical variables as murmur-grades may allow a murmur-grade value to be determined from a calculated average of a plurality of single-digit integer classifications, e.g. by calculating the mean of two or more standard Levine scale murmur-grade values. In embodiments where the murmur-grade values are determined by two or more clinicians before being given as input to the computer software, this may advantageously allow differing assessments from different clinicians to be aggregated in a manner which captures any lack of consensus.

In a set of embodiments, two, three or four murmur-grade values are received, each murmur-grade value being associated with a different one of an aortic, pulmonary, tricuspid or mitral auscultation point. Each auscultation position may be a standard auscultation point. Each may be a location at which the heart sounds which can be heard or recorded emanate principally from a respective one of the aortic valve, pulmonary valve, tricuspid valve, or mitral valve.

The plurality of murmur-grade values may have been determined by a clinician, e.g. comprising the clinician listening to the heart using a stethoscope at each of the respective auscultation positions, and assigning a murmur-grade value to each of the auscultation positions. However, in a set of embodiments, at least one, or all, of the murmur-grade values are generated by a second trained software model, referred to herein as a murmur-grading software model. The computer software disclosed herein may implement (i.e. provide) this murmur-grading software model. In a set of such embodiments, a plurality of sound recordings of the heart may be received, each sound recording being captured by a microphone positioned at a different respective one of the auscultation positions (e.g. at a different standard auscultation point). Each sound recording may be provided as input to the trained murmur-grading software model, which may be operated to output the murmur-grade value associated with the respective auscultation position. The murmur-grading software model may process each sound recording independently. The murmur-grade values may be received internally within the processing system (e.g. being received by the first trained software model from the murmur-grading software model, with both models executing on the same processing system) or they may be received from outside the processing system. The murmur-grade values may be read from a digital memory (e.g. from RAM) or may be received in any other way.

Any combination of sources may be used to provide the plurality of murmur-grade values associated the different respective auscultation positions. Zero, one or more of the plurality of murmur-grade values may be generated from sound recordings using a murmur-grading software model as described as above, and zero, one or more of the plurality of murmur-grade values may be determined by a clinician.

In a set of embodiments, sound recordings may be captured at any or all of the auscultation positions. The sound recordings may be captured sequentially, such that each recording is captured over a different non-overlapping period of time. Although different in time, it may be beneficial if the recordings are taken close to each other in time. In some embodiments, the same microphone is used for capturing each sound recording, while in other embodiments a plurality of microphones (e.g. transducer elements) may be used. In embodiments where at least one sound recording is received, each sound recording may span any number of cardiac cycles—e.g. two, ten, a hundred or more.

In a set of embodiments, the first trained software model is a regression model. It may be a linear regression model. The regression model may be multi-variate, such that the value indicative of valve health depends on at least two variables (e.g. on four or eight variables, which may be four murmur-grade values and optionally four quality indicator values). The murmur-grade at two or more different auscultation positions may be used by the first trained software model to generate the value indicative of valve health. In a set of embodiments, the regression model comprises at least one second-order or third-order term.

In a set of embodiments, a plurality of quality indicator values is received and provided as input to the first trained software model. Each quality indicator value may be associated with a different respective one of the auscultation positions. A respective quality indicator value may be received for each of the plurality of received murmur-grade values. The value indicative of valve health generated by the trained software model may depend, at least in part, on at least one of the quality indicator values. Each of the plurality of quality indicators may represent a confidence measure for the murmur-grade value associated with the respective auscultation position. Advantageously, this allows the processing of the different murmur-grade values to be adjusted depending on whether the murmur-grade values used as input is likely to be accurate. In some embodiments, the first software model may apply a higher weighting to a murmur-grade value associated with a first position than would otherwise be applied in response to determining that the quality indicator value associated with a second position is below a threshold value.

In a set of embodiments, each quality indicator is a binary measure. The first software model may use each binary measure to determine whether or not to use the murmur-grade value at the respective associated auscultation position when generating the value indicative of the health of the heart valve. In embodiments where at least one sound recording is received for an associated auscultation position, each quality indicator may indicate a quality of the associated sound recording. A first binary quality indicator value may be assigned to a sound recording when the sound recording is determined to contain noise, and a second binary value may be assigned when the sound recording is determined not to contain noise. This may be used to cause the first trained software model to discard a murmur-grade value generated by the murmur-grading software model for any auscultation position that is associated with the first binary quality indicator value, when generating the value indicative of valvular health of the heart.

In a first set of embodiments, the plurality of quality indicator values may be generated by the trained murmur-grading software model. In a second set of embodiments, the plurality of quality indicator values may be provided by a human operator (e.g. by a person who listens to the sound recordings). Providing human-assessed quality indicator values to the first trained software model may be beneficial in embodiments in which the trained murmur-grading model is not arranged to estimate quality indicator values.

Although, in some embodiments, the output from the trained regression model may depend on received quality indicator values, this is not essential in all embodiments. For instance, when using a murmur-grading software model that has been trained to be robust to noisy data (e.g. by being trained on sound recordings having a mix of high and low quality indicator values), the trained regression model may not need to take account of the quality of the sound recording when predicting a murmur-grade value. Thus, some embodiments do not pass quality indicator values from the murmur-grading software model to the trained regression model.

In a set of embodiments, the first trained software model has been trained using supervised learning on training data comprising training tuples, each comprising a plurality of murmur-grade values associated with different auscultation positions for a respective heart, and a ground-truth (i.e. target) value indicative of the health of a valve of the respective heart. The training tuples may identify with which auscultation position each murmur-grade value is associated. The ground-truth values may all relate to a same type of valve, which may be the aortic valve. The murmur-grade values may be determined by a clinician or may be output by a trained murmur-grading software model as disclosed herein. The ground-truth values may be AVPG values, which may be determined by an echocardiogram. Each training tuple may additionally comprise a quality indicator value, as disclosed herein. The quality indicator values may be determined by a human operator or may be output by a trained murmur-grading software model as disclosed herein.

From another aspect, the invention provides a method of training a software model, the method comprising:
receiving training data comprising training tuples, each training tuple comprising a plurality of murmur-grade values associated with different auscultation positions for a respective heart and a ground-truth value indicative of the health of a valve of the respective heart; and
using the training data to train the software model for generating values indicative of health of a heart valve.

Some embodiments of the computer-implemented methods for assessing valve health disclosed herein, may comprise training a first software model to generate the first trained software model. Some embodiments of the computer-implemented methods may comprise training a second software model to generate a trained murmur-grading software model as disclosed herein. However, in other embodiments, the trained first and/or second software models are pre-trained.

The first trained software model may have been trained by fitting a multi-variate regression model to the training data.

In a set of embodiments where a murmur-grading software model is used, the murmur-grading software model comprises a neural network. In such embodiments, the model may have been trained using supervised learning on training data comprising training tuples, each comprising a sound recording of a respective heart and a ground-truth murmur-grade value for the sound recording. The murmur-grading software model may be arranged to receive an identification of an auscultation position at which the sound recording was captured. However, in other embodiments, the murmur-grading software model does not know from which auscultation position each sound recording was obtained—i.e. it may be configured to process each sound recording without using information (supplemental to the actual sound recording itself) that identifies which auscultation position the sound recording was obtained from. This may enable the murmur-grading model to be trained more efficiently for a given number of training recordings. The ground-truth murmur-grade values may be determined by one or more clinicians who listen to the sound recordings. Each may be an average (e.g. mean) value of a plurality of Levine-scale grade values determined by a respective plurality of clinicians. Each training tuple may additionally comprise a ground-truth quality indicator value, as disclosed herein, which may be determine by a human operator who reviews the sound recordings.

The murmur-grading software model may be trained independently from the first software model. Partitioning the computer software into two independent software models, with the first receiving murmur-grade values generated by the other, has been found to facilitate efficient convergence of both models, while enabling the first software model to output clinically-useful values of heart valve health (e.g. AVPG values). It may result in a higher probability of the software converging on good parameter configurations, in comparison to training a single model end-to-end to predict a value such as an AVPG value directly from an input set of sound recordings.

Nevertheless, while aspects of the invention comprise the first trained software model and a distinct murmur-grading software model, this is not essential in all aspects of the invention.

From a further aspect, the invention provides a computer-implemented method for assessing health of a heart valve of a heart of a subject, the method comprising:
receiving, as input into a trained software model, a plurality of sound recordings of the heart, each sound recording being captured by a microphone positioned at a different respective auscultation position (e.g. at a different standard auscultation point);
operating the trained software model to generate a value indicative of health of the heart valve; and
outputting the value indicative of health of the heart valve from the trained software model.

This aspect extends to computer software comprising instructions for performing the method, and to a computer system configured (e.g. programmed) to perform the method.

This trained software model may process each sound recording independently. It may comprise any appropriate feature or features of the first trained software model and/or the murmur-grading software model disclosed herein. It may be trained end-to-end. Alternatively, it may comprise a first model and a second model that are trained independently, wherein the first model is configured to receive the plurality of sound recordings and to output intermediate data to the second model, and wherein the second model is configured to receive the intermediate data from the first model and to output the value indicative of health of the heart valve. The intermediate data may comprise murmur-grade values in some embodiments, but this is not essential in every embodiment of this aspect.

Any of the computer software disclosed herein may be provided on a non-transitory computer-readable medium, which may be a solid-state memory or a magnetic or optical storage medium. It may be stored in volatile memory (e.g. RAM) or non-volatile memory (e.g. flash memory, EEPOM, ROM). The software may be written in any language, e.g. C, C++, Java, etc. It may be compiled into machine code. The computer system may comprise one or more microprocessors. It may comprise memory, buses, internal peripherals, input/output peripherals, network interfaces, a power supply, etc. A software model as disclosed herein may be trained on one computer system and the resulting trained software model (or a copy thereof) may be executed on a different computer system.

Features of any aspect or embodiment described herein may, wherever appropriate, be applied to any other aspect or embodiment described herein. Where reference is made to different embodiments or sets of embodiments, it should be understood that these are not necessarily distinct but may overlap.

### BRIEF DESCRIPTION OF THE DRAWINGS

Certain preferred embodiments of the invention will now be described, by way of example only, with reference to the accompanying drawings, in which:

FIG. 1 shows a schematic view of a human chest indicating four standard auscultation points;

FIG. 2 shows a schematic overview of a clinician capturing a sound recording from the body of a subject using an electronic stethoscope;

FIG. 3 shows a schematic overview of a two-part model for health of a heart valve including a trained neural network model and a trained regression model in accordance with embodiments of the present invention;

FIG. 4 shows a schematic overview of the trained regression model of FIG. 3;

FIG. 5 shows a schematic overview of the process of collecting training data needed to train one or both of the trained neural network model and the trained regression model of FIG. 3 in accordance with embodiments of the present invention;

FIG. 6 shows a schematic overview of the structure of the neural network of FIG. 3;

FIG. 7 shows a schematic overview of the training process for the neural network of FIG. 6;

FIG. 8 shows a schematic overview of the steps between raw heart-sound (HS) signal input and the murmur-grade prediction using the neural network of FIG. 6;

FIG. 9 is a graph showing the area under curve (AUC) performance metric for the trained regression model of FIG. 4 as a function of cut-off threshold compared to an aortic-only prediction model;

FIG. 10 is a graph showing the area under curve (AUC) performance metric for the trained regression model of FIG. 4 as a function of cut-off threshold compared to a pulmonic-only prediction model;

FIG. 11 is a graph showing the area under curve (AUC) performance metric for the trained regression model of FIG. 4 as a function of cut-off threshold compared to a tricuspid-only prediction model;

FIG. 12 is a graph showing the area under curve (AUC) performance metric for the trained regression model of FIG. 4 as a function of cut-off threshold compared to a mitral-only prediction model;

FIG. 13 is a graph showing the area under curve (AUC) performance metric for the trained regression model of FIG. 4 as a function of cut-off threshold compared to a prediction model using the sum of the aortic and pulmonic predictions;

FIG. 14 is a graph showing the area under curve (AUC) performance metric for the trained regression model of FIG. 4 as a function of cut-off threshold compared to a prediction model using the maximum of the aortic and pulmonic predictions;

FIG. 15 is a graph showing the area under curve (AUC) performance metric for the trained regression model of FIG. 4 as a function of cut-off threshold compared to a prediction model using the maximum of the predictions at all positions;

FIG. 16 is a graph showing the area under curve (AUC) performance metric for the trained regression model of FIG. 4 as a function of cut-off threshold compared to a prediction model using the sum of the predictions at all positions;

FIG. 17 is a graph showing receiving operator characteristic curves illustrating the ability of the neural network of FIG. 6 to detect murmur-grades values.

### DETAILED DESCRIPTION

A valuable metric which is known to be used as an indicator of health of a heart valve is the time-averaged mean pressure gradient across the valve, which can be measured using an echocardiography examination. In particular, the mean pressure gradient across the aortic valve is important metric used to identify aortic stenosis (AS), which is a narrowing of the aortic valve opening that typically gives rise to an increased pressure difference across the aortic valve. Although some embodiments described herein focus on the aortic valve, it should be understood that the pressure gradient across any of the valves in the heart may be informative for assessing the valvular health of the heart.

However, referral to an echocardiography examination can be time consuming and expensive. Instead, it is common practice for clinicians to perform an initial assessment of valvular heart health by listening to sounds emanating from the heart with a stethoscope. In particular, the presence of an audible murmur can indicate abnormal functioning of a valve. This information gathered from examination with a stethoscope is more readily available than a measurement of a valve pressure gradient obtained through echocardiography. In a clinical setting, it is common practice for a subject to be referred for an echocardiogram if a clinician determines that a significant murmur can be heard.

The applicant has appreciated that the mean aortic valve pressure gradient (AVPG) may be estimated with a good degree of accuracy by processing a plurality of murmur-grade values associated with different respective auscultation points around the heart in a trained software model, thereby providing more insight into valvular heart health without requiring an echocardiogram. The applicant has found that using a plurality of murmur-grade values provides greater insight for assessing valvular hearth health than can be obtained by interpreting murmur-grade values for individual auscultation points around the heart in isolation. It may be that, if an AVPG value estimated by the software model is elevated, the subject can then be referred for an echocardiogram to determine if a diagnosis of aortic stenosis should be made.

Murmur-grade values associated with different valves of the heart can conventionally be determined by analysing heart sounds which can be heard or recorded at different locations around the exterior of the chest, known as auscultation points or positions. Sounds at these different locations may better inform an understanding of the behaviour of the subject's heart. When a clinician is aiming to examine a subject thoroughly with a view assessing heart health, it is known that there are four principal auscultation points (not counting Erb's point) which should be examined.

Typically, a clinician will listen to the heart at each of the auscultation points with a stethoscope, and assign a murmur grade to each point based on a qualitative assessment of the sound.

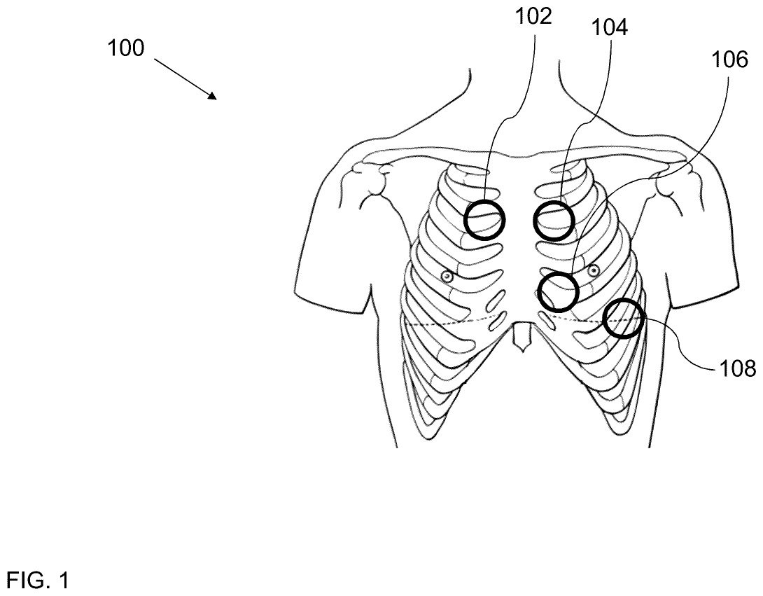

FIG. 1 shows a schematic view of a human chest 100, indicating the four standard auscultation points 102, 104, 106 and 108. These locations are adjacent the expected positions of the aortic valve (location 102), pulmonary valve (location 104), tricuspid valve (location 106) and mitral valve (location 108). In some of the examples described herein, the received murmur-grade values are associated with these four different auscultation points. However, in other embodiments the software model may be trained for using more or fewer auscultation points and/or one or more non-standard auscultation positions.

In the set of embodiments described herein, the heart sounds examined are of the human heart; however, it should be understood that the methods disclosed herein may be used to process heart sounds emanating from the heart of any animal subject.

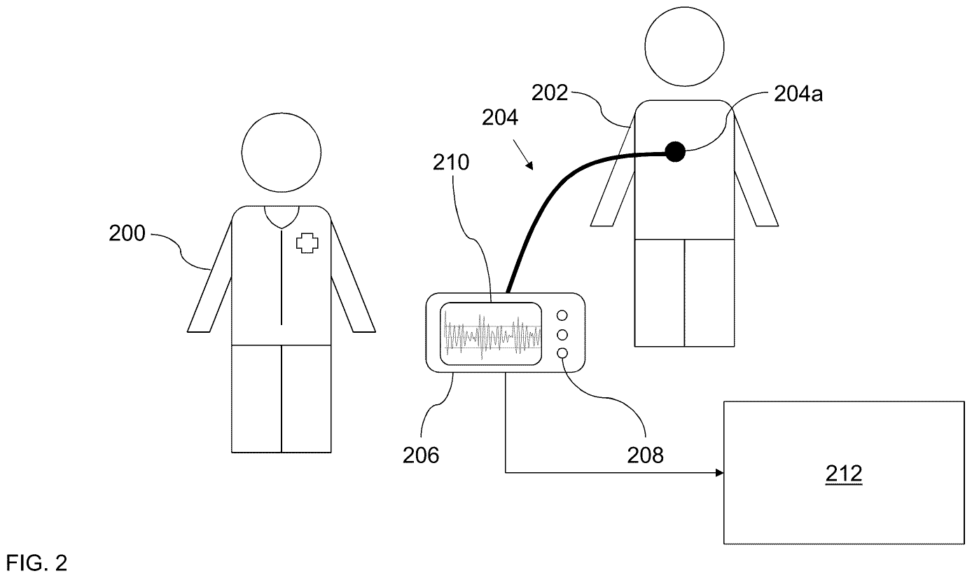

FIG. 2 shows a schematic overview of a clinician 200 capturing a sound recording from each of the four auscultation points 102-108 on the chest of a subject 202 using an electronic stethoscope 204. The clinician 200 places the chest piece 204a of the electronic stethoscope 204 (which contains a microphone) at a location on the exterior of the chest of the subject 202, and controls a recording system 206 using controls 208 and a display 210 such that a recording of sound emanating from the heart, spanning a given length of time (e.g. ten seconds), is captured for that location. The captured sound recording can be digitised by the recording system 206 and subsequently be processed by a computer system 212 embodying the invention. The electronic stethoscope 204 may be physically connected to the computer system 212, or it may be able to communicate by wireless link. The computer system 212 could alternatively be integral with the recording system 206. The computer system 212 could alternatively be remote from the clinician 200 and subject 202—e.g. in a server farm in a different town or country. In some embodiments, the sound recordings may be stored on a data storage medium and processed by the computer system at a different time and/or location from when they were captured. The subject 202 need not necessarily be present when the sound recordings are analysed by the computer system 212.

In some embodiments, the chest piece 204a may comprise multiple microphones and be able to capture sound recordings simultaneously at multiple auscultation positions, but in this example the clinician 200 moves the chest piece 204a between auscultation points on the chest over time, such that each sound recording is made over a different time period.

The clinician may then listen back to the recordings in order to assign a murmur-grade to the sound captured at each auscultation point, or the recordings may be processed further in order to determine a murmur-grade using computer-implemented software (e.g. using a trained murmur-grading software model, as disclosed herein). The computer system 212 (e.g. a workstation or a laptop computer or a remote server) may contain a processor and a memory storing software for execution by the processor for performing some or all of the processing operations disclosed herein. It may have its own input and output interfaces, such as a display screen.

Although a murmur-grade may be assigned to the sound captured at each auscultation point by a clinician, in some embodiments the recordings are instead processed in order to determine a murmur-grade using computer-implemented software executing on the computer system 212.

In some embodiments, the computer software comprises two main parts: a trained neural network model, and a trained regression model. First, it uses a trained neural network to predict murmur grades as continuous numerical variables (i.e. including values having non-zero decimal components, rather than only single-digit integers), using audio gathered from each of four auscultation positions. Then, it provides each set of predicted murmur grades as input to a multivariable trained linear regression model that is trained to model the relationship between the predicted murmurs and the time-averaged mean aortic valve pressure gradient (AVPG).

The models are trained independently (although potentially using the same or overlapping training data), but when the two trained models are combined, the overall model can provide an end-to-end algorithm which attempts a numerical prediction of mean AVPG based solely on audio data collected with a stethoscope 204 as input.

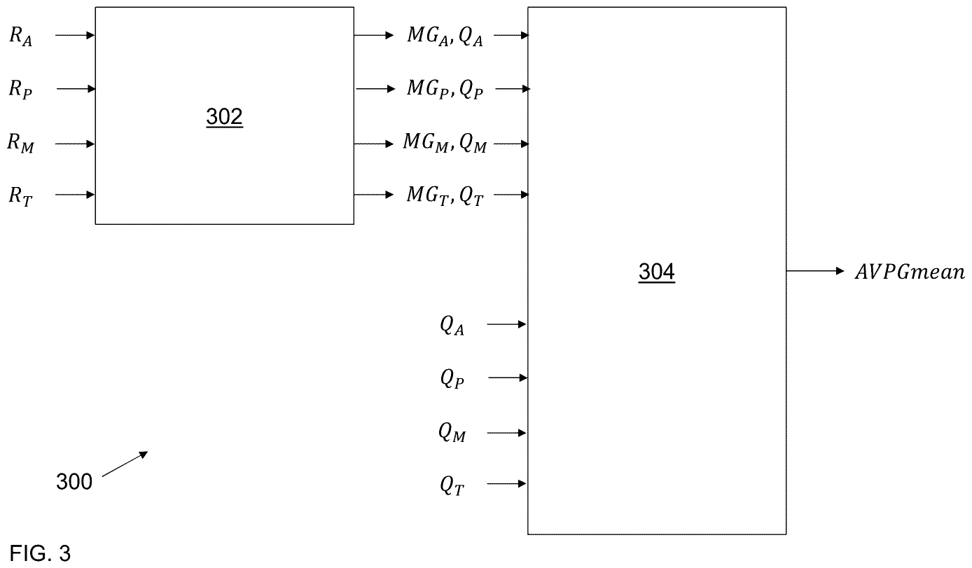

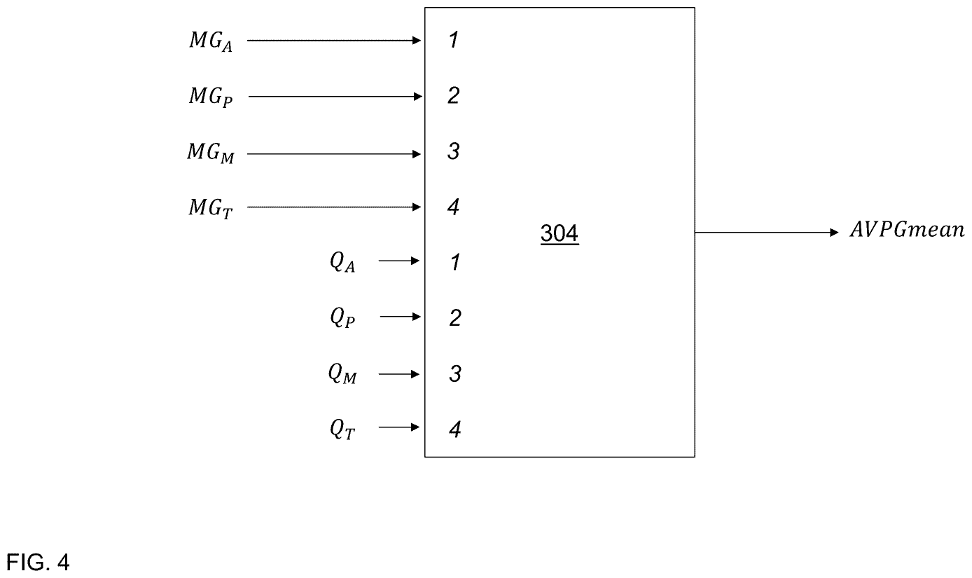

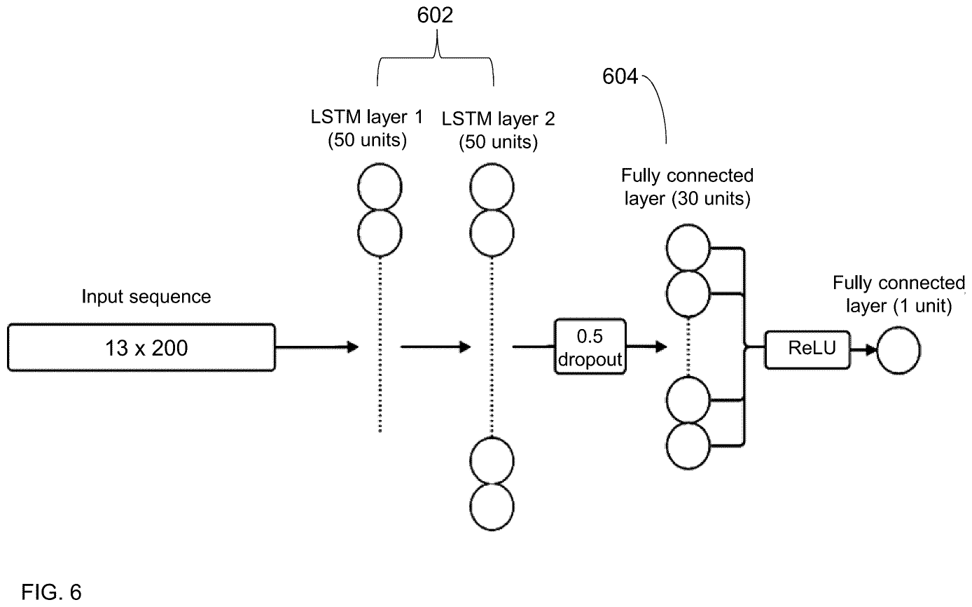

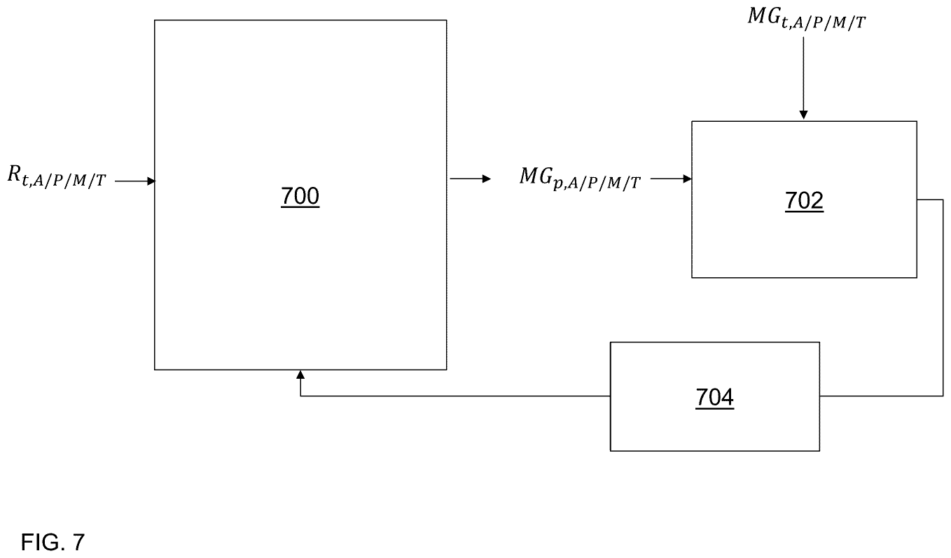

The two-part model 300 is shown schematically in FIG. 3. First, a set of four sound recordings R_{A}, R_{P}, R_{M} and R_{T}, captured at the aortic, pulmonary, mitral and tricuspid auscultation points respectively, are given as input to a trained murmur-grading model 302. The murmur-grading model 302 is used to generate four murmur-grade value predictions MG_{A}, MG_{P}, MG_{M} and MG_{T}, respectively associated with each auscultation position, as will be described in more detail with reference to FIGS. 6 and 7. These murmur-grade values, along with respective quality indicator values or measures Q_{A}, Q_{P}, Q_{M} and Q_{T}, which may have been generated by the murmur-grading model 302 or determined by a human annotator, are then provided to the trained regression model 304 as input, which is operated to generate a mean AVPG prediction AVPGₘₑₐₙ as will be described in more detail with reference to FIG. 4. The AVPG value output from the model 304 may be displayed to the clinician 200 on a display of the computer system 212 for consideration and/or may be stored in a memory of the computer system 212 for later analysis by the same or a different clinician and/or may be further processed by the computer system 212 or another computer system, e.g. to generate an automatic referral letter to an echocardiogram clinic.

The trained linear regression model 304 is shown schematically in FIG. 4. The trained model 304 takes an aortic murmur-grade (MG) value MG_{A}, a pulmonary murmur-grade value MG_{P}, a mitral murmur-grade value MG_{M} and a tricuspid murmur-grade value MG_{T} as input. The inputs are ordered, such that the model 304 knows which auscultation position is associated with each input. The trained model 304 may also receive a quality measure Q_{A}, Q_{P}, Q_{M} and Q_{T} associated with the aortic, pulmonary, mitral and tricuspid positions respectively as input. These could each have a range of possible values, but in some examples they are each just a binary value, indicating whether or not the associated sound recording contains significant noise (i.e. artefacts). The output of the trained model 404 is a prediction of the mean Aortic Valve Pressure Gradient, AVPGmean. The output could be expressed according to a standard scale (e.g. using SI units), but this is not essential.

The trained linear regression model 304 is a multivariate model, with the murmur-grade values MG_{A}, MG_{P}, MG_{M} and MG_{T} and the quality measures Q_{A}, Q_{P}, Q_{M} and Q_{T} at the different auscultation points as variables of the model.

Providing the quality measures Q_{A}, Q_{P}, Q_{M} and Q_{T} as input to the model 304 enables the reliability of the murmur-grade value associated with each auscultation point to be taken into account in the mean AVPG prediction. In some examples, the trained linear regression model 304 may adjust the coefficients applied to the terms in the regression model if any of the recordings used to determine a murmur-grade are deemed to be noisy (e.g. having a quality indicator below a threshold level). This means that, in the case that one auscultation position is affected by noise, terms in the regression model that depend on more reliable noise-free murmur-grade values at other positions can be weighted more heavily, whilst the weighting of terms that depend on the noisy murmur-grade values could be decreased. Indeed, if a recording is prohibitively noisy, the contribution of the murmur-grade value from that recording might be discounted altogether (e.g. weighted to zero).

Before being used to predict mean AVPG from sets of murmur-grade values, the regression model 304 (or a prototype model, from which the model 304 and any number of copies are derived) is trained using a training data set in order to determine the terms to include in the multi-variate regression and the coefficients which should be used with each term. The training set includes data collected by clinically examining a large number of different hearts. The neural network in the murmur-grading model 302 (or a prototype for the model 302) is also trained before being used for inference in the computer software. The murmur-grading model 302 may be trained using the same training data as the regression model 304, or using different training data.

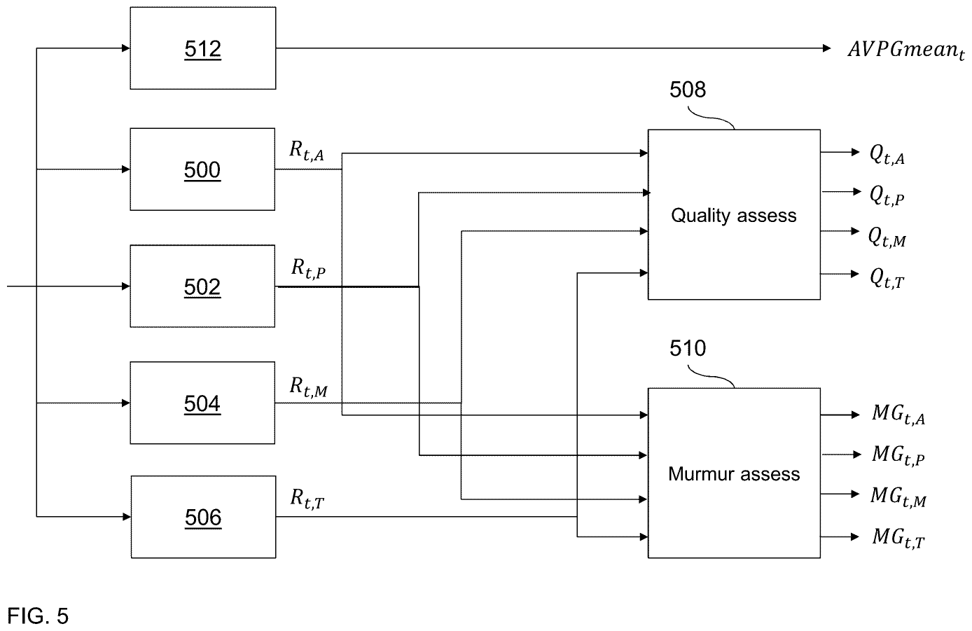

For each heart in the training set, FIG. 5 shows the data collection process used to generate the clinical information needed for training the models 302 and/or 304. For each heart, a clinician records sounds from four different auscultation points in steps 500, 502, 504 and 506—at the aortic, pulmonary, mitral and tricuspid positions respectively—to generate training sound recordings R_{t,A}, R_{t,P}, R_{t,M} and R_{t,T}. Each of these recordings is then assessed for quality in step 508, where a clinician (or more than one clinician) assign(s) a quality value to each of the captured sound recordings. This quality assessment step 508 outputs training quality indicator values Q_{t,A}, Q_{t,P}, Q_{t,M} and Q_{t,T}.

Each recording is also assessed to determine a corresponding murmur-grade value in step 510. When training the regression model 304, this step may be completed by the trained murmur-grading model as described further with reference to FIGS. 6 and 7. Alternatively, and also when generating training data for training the murmur-grading model 302, one or more clinicians assign a murmur-grade value (which could be an averaged value across multiple clinicians, e.g. a mean murmur-grade) to each recording. Whether manually annotated or computer-annotated, a set of training murmur grades—MG_{t,A}, MG_{t,P}, MG_{t,M} and MG_{t,T}—is determined.

As well as processing each of the sound recordings to gather training murmur-grade values and quality indicator values, each heart is also examined using an echocardiogram in step 512 to measure a training mean aortic valve pressure gradient, AVPGmeanₜ. If the mean pressure gradient across any other of the valves (e.g. the pulmonary valve) is of interest, this could instead or additionally be measured. In a set of embodiments described herein, only the aortic mean pressure gradient is calculated. This may be useful for assessing heart health generally, or for determining whether to refer a patient for further investigation to determine if aortic stenosis is present.

Once murmur-grade predictions, Xᵢ, and quality indicator values (representing noise), Nᵢ, are determined for each of the n (e.g. four) positions, they are aggregated in a way that produces an optimal predictor of mean AVPG through use of a linear regression model.

The training process for the regression model 304 seeks to converge on a model which best predicts an AVPG value, as a continuous target, from a set of murmur-grade value (and optionally also quality values).

An exemplary model 304 described herein was fitted using the predicted murmur-grades output by a murmur-grading networks 302 that was trained on both clean and noisy data.

Model selection for the regression model 304 may be carried out through stepwise inclusion and exclusion of terms, with decisions to keep or discard a change guided by model-fitting-parameters such as parameter p-values and some goodness of fit measure, such as the Bayesian Information Criterion (BIC) or adjusted R-squared.

In the example described herein the search scope was be limited by allowing only models that include up to third degree terms (including interaction terms) such as X_{A}³, and X_{A}*X_{M}². Interaction terms may be considered that allow the model 304 flexibility in terms of how the noisiness of the samples should be handled, such as X_{A}*N_{P} for re-adjusting the weight of X_{A} in the case where the pulmonic position has been deemed un-usable (either by human or computer-automated evaluation).

Transforms of the independent variable in the modelling process may be considered, such as T(AVPGₘₑₐₙ)=\sqrt{AVPGmean}, as linear increase in murmur-grade need not correspond to linear increase in mean AVPG. Additionally, a concave transform, such as the square root, will reduce the risk of overfitting to a small set of very extreme values, since it reduces the range between the smallest and largest values and thus reduces the effect of extreme values in the model fitting. The prediction of the model 304 can be transformed back to the original scale before being output, although this is not essential. In experimental testing, described below, the output was transformed back before computing performance metrics; for instance, if T(x)=\sqrt{x} then the inverse T⁻¹(x)=x² would be applied before measuring the size of the prediction errors.

In some embodiments, the model 304 that was selected, using a model selection process, with fitted parameters inserted, is:

$$\sqrt{AVPGmean} = 1.85 + 0.13 \cdot {X}_{A}^{3} + 0.2 \cdot {X}_{P} + 0.22 \cdot {X}_{T} - 0.09 \cdot {X}_{T}^{2} + 0.07 \cdot {X}_{M}^{2} + 0.11 \cdot {X}_{A} \cdot {X}_{T} + ϵ .$$

where A, P, T, M refers to the aortic, pulmonary, tricuspid and mitral position, and X variables denoted i refer to predicted murmur-grade in position i.

The following table shows estimated coefficient values, determined to a higher precision than the two decimal places shown in the equation above, for the linear regression model 304. These were calculated by fitting predicted murmur-grades generated by the murmur-grading model 302 that was trained on both clean and noisy audio.

| Variable | Coefficient | p-value |
|---|---|---|
| X_{A}³ | 0.02762 | <1e−5 |
| X_{P} | 0.20378 | <1e−5 |
| X_{T} | 0.21798 | <1e−5 |
| X_{T}² | −0.09310 | 0.0002 |
| X_{M}² | 0.07147 | <1e−5 |
| X_{A} * X_{T} | 0.10518 | 0.001 |

Except for the 2nd degree Tricuspid term

$${X}_{T}^{2} ,$$

the coefficients are all positive, indicating that in general, the more auscultation positions have murmurs, the higher the chance of a high mean AVPG. The high significance of each positions term indicates that each murmur-prediction does contribute significantly to the prediction of the mean AVPG, and that it may be beneficial in some embodiments that no position is discarded when attempting an AVPGₘₑₐₙ prediction. In particular, it is surprising to see the high significance of the tricuspid and pulmonic positions, as these are typically not considered by physicians in the context of listening for aortic valve murmurs.

One interaction term is included, being a term reflecting a positive interaction between the aortic and tricuspid murmur. The applicant has determined that a high aortic murmur combined with a high tricuspid murmur can be particularly suggestive of a high mean AVPG.

Note that for the specific example of the selected model described above, no terms including quality indicator values Nᵢ were found to be significant, and thus were not included in the selected model 304. The applicant has appreciated that this is the case at least partly because the selected model 304 was fitted using murmur-grade values which were determined by a murmur-grading software model which was trained on both noisy and clean sound recordings. Accordingly, the quality indicator values may have been found to be not significant because the murmur-grade predictions from the murmur-grading model were more robust to noisy recordings in the first place. However, quality-indicator values could be found to be significant in other embodiments and may then be used to determine the output of the trained regression model—e.g. appearing as binary or linear weights for one or more terms.

The applicant has appreciated that a model 304 trained to predict mean AVPG from a plurality of murmur-grade values can provide a valuable, new, easily-accessible way of gaining insight into heart health, given that audio data for the heart is more readily and cheaply available than echocardiography data.

Embodiments where the recordings are processed in order to determine a murmur-grade using the murmur-grading software model 302, which is trained to assign a murmur grade to sound recordings at each of the auscultation positions, are described in more detail below with reference to FIGS. 6-8.

The structure of an exemplary neural network used for the murmur-grading model 302 is shown in FIG. 6. For the network-architecture a 2-layer (50 neurons each) long-short-term-memory (LSTM) network 602 is provided, followed by a fully connected layer 604 consisting of 30 neurons. The network was trained to predict murmur-grade as a continuous variable (i.e. able to predict non-integer values, as well as integer values).

Before being used to predict murmur-grade values MG; from sound recordings Ri, the murmur-grading model 302 is trained using a training data set. The training set includes data collected by clinically examining a large number of different hearts (e.g. as described above). The training process is shown in FIG. 7.

The murmur-grading model 302 was trained using sound-recordings from all four auscultation points, effectively treating each subject's set of recordings as four independent observations (i.e. agnostic to the position at which the recording was taken). This can allow for faster convergence of the model 302 for a given number of training recordings.

Accordingly, in each training iteration, a training recording R_{t,i}, obtained from any position i, is given as input to the untrained murmur-grading neural-network model 700, which predicts a murmur-grade value MG_{p,i} for the recording (without using knowledge of what auscultation position it was taken from). The predicted murmur-grade value MG_{p,i} is then compared to the training murmur-grade value MG_{t,i} which was determined by a clinician's annotation as described with reference to FIG. 5. A loss function is evaluated by a loss-function module 702 based on the difference between the annotated training murmur-grade MG_{t,i} and the predicted murmur-grade MG_{p,i}. A model-updating module 704 then updates the weights of the neural network based on the output of the loss function, i.e. using backpropagation.

The prediction target is murmur grade as a continuous variable. The grading scale used in the embodiments described herein is the Levine scale, which grades perceived murmurs on a scale from 1-6 based on features such as loudness and audibility of the murmurs, but modified to allow fractional components within this scale. Note that some of these features are lost when listening to pre-recorded audio, such as thrill, and whether or not the murmurs can be heard with the stethoscope not touching the chest. In general, any system of murmur grading the recordings in a way that results in a numerical variable that correlates well with the mean AVPG could be used as a training target.

The model 700 is trained on the murmur-grade values as a continuous numerical variable, rather than splitting it into distinct classes of murmurs. This choice was made in order to make more efficient use of the information in the labels, as there is always loss of information when reducing a graded or continuous variable down to a binary variable, or integer classes. Furthermore, using a numerical variable not limited only to integer values for the training target allows the murmur-grade label to be determined by aggregating assessments from more than one clinician. For example, the mean of two single-digit integer murmur-grades assigned using the Levine scale may be calculated, allowing differing assessments from different clinicians to be aggregated in a manner which captures any lack of consensus.

Once training is complete, any number of trained models 302 can be cloned from the model 700, for operation in inference mode in systems as disclosed herein.

Separating out the prediction of a murmur grade from the prediction of the mean AVPG value by training two independent models 302 and 304 for each respective task allows the murmur-grading neural network 302 to be trained on the presence of a murmur only. The applicant has appreciated that, with sound recordings as input, the murmur grade is an easier target to train on than mean AVPG, and so the murmur-grading model 302 therefore produces more reliable results than if it were trained directly on mean AVPG. Murmur grade is determined by features that are actually expressed in the network's training input (i.e. the sound recordings), whereas the mean AVPG represents a background state which may or may not be expressed in the sound signal. The applicant has appreciated that this results in a lower probability of the network 302 converging on good parameter configurations, and that this improvement in training efficiency is due to a clear relationship between what the network can observe in the input (expressed features) and the loss-value associated with that sample.

Furthermore, the expression of the mean AVPG will tend to vary between auscultation positions which means that a naive approach of training a network directly on sound recordings and ground-truth mean AVPG values, without regard for the position that each sound recording was collected from, is unlikely to result in efficient training. With the murmur-grade as a target, however, the relationship between label and label-expression should be consistent across the auscultation positions.

The training of the regression model 304 may be performed after the murmur-grading model 302 has been trained, using any suitable regression fitting training technique (e.g. as described in more detail in the example implementation below).

In addition to improving the training efficiency and convergence of each of the two models 302, 304, the use of murmur-grade values as an interface between the two models 302, 304 may also improve the explainability and acceptability of the system 300 to users.

Considering murmur-grade values from a plurality of auscultation positions for assessing the health of a particular valve by a software model 304 has been found to work particularly well. The following sections describe, in greater detail, some particular exemplary implementations and some experimental validation results that have been obtained from them. These demonstrate the utility of the approaches disclosed herein.

### Example Implementations and Experimental Validation

### Study Design

The Tromsø study is an ongoing population-based prospective study that was initiated in 1974, in the municipality of Tromsø, northern Norway. The 7th and most recent survey was carried out in 2015 and 2016, which provided the data used in this study. 32 591 people received invitations to participate in the Tromsø 7 study, of which 64.7% participated. All inhabitants of Tromsø aged 40 or older were invited to participate in the study, and the Tromsø study is the largest study of its kind in Norway. The age of the participants ranged from 40-99. A subset of the participants underwent echocardiography examination (n=2340), and physical exams which included cardiac auscultation (n=2409). Altogether, 2132 participants had both echocardiography and heart sound recordings.

### Heart-Sound Recording

We attached a microphone (Sennheiser MKE 2-EW) inside the tube of a stethoscope (Littmann classic II) 10 cm from the chestpiece. The microphone was connected to a wireless system (Sennheiser EW 112-P G3-G) that transmitted the sound signal to a computer via an external sound card (Scarlett 212, Focusrite Audio). We recorded heart sounds for 10 seconds. Audio files were recorded in “.wav” format in a single monophonic channel with a depth of 16 bits at a rate of 44, 100 Hz. Participants were sitting in a chair and were asked to breathe normally. For each participant recordings were collected in four locations: aortic (2nd intercostal space, right parasternal line), pulmonic (2nd intercostal space, left parasternal line), tricuspid (5th intercostal space, left parasternal line), and mitral positions (5th intercostal space, left mid-clavicular line), which in the following will be referred to as positions 1-4 when convenient.

### Heart-Sound Annotation

The heart sound recordings were independently annotated by two GPs. This training was reinforced at intervals throughout the annotation process when the two annotators discussed disagreements and pursued consensus on the presence of murmurs. The annotators were blind to the echocardiography results and other information about study participants during the heart sound annotation.

Each recording was annotated by listening to each recording while looking at a visualization provided by the spectrogram. Any perceived murmurs were graded on a scale of 1-6, and each recording was annotated as systolic, diastolic, or noisy. The annotators were blind to any information about the participants beyond the heart sound recording. In total, 2129 participants had annotated heart sound recordings, raw data from echo-exams, and were annotated with a severity grade. Of these, 5 were removed due to corrupted audio-files, resulting in a total of 2124 participants, and 8496 annotated audio files (with 1416 minutes of recorded audio).

For algorithm training, we took the average murmur-grade to represent each position. In the following, this value is referred to simply as the murmur-grade. By this convention, the data-set contained a total of 465, 280, 303, and 196 cases of murmur-grade>0 in positions 1-4 respectively, yielding a total of 1244 audio recordings for which at least one annotator perceived a murmur.

Taking the average murmur grade from the two clinicians annotating the recording was advantageous for improving the performance of the model.

### Echocardiography

All echocardiogram examinations were performed according to the American Society of Echocardiography's Guidelines using a GE Vivid E9 (GE Medical, Horten, Norway) ultrasound scanner. The examination was performed by an echocardiogram technician before the heart sounds were annotated by an experienced cardiologist. AS severity was graded on a scale of 0-3 (absent, mild, moderate and severe). AS was graded using the aortic-valve-mean-pressure-gradient (AVPGₘₑₐₙ), using cut-off values 15 mm Hg (mild), 20 mm Hg (moderate) and 40 mm Hg (severe).

### Algorithm Development

The murmur-detection algorithm was trained using HS-recordings from all four auscultation positions, effectively treating each individual's set of recordings as four independent observations. For network-architecture we used a 2-layer (50 neurons each) long-short-term-memory (LSTM) network, followed by a fully connected layer consisting of 30 neurons. The network was trained to predict murmur-grade as a continuous variable. As the dataset was highly imbalanced, we resampled from the positive class (defined in this context as all observations with murmur-grade ≥1) until the positive and negative classes were of equal size. For dimension reduction of the audio-recordings, we used the 13 first Mel-frequency-cepstral-coefficients (MFCC).

Each recording was segmented into six overlapping (50%) blocks, each consisting of four cardiac cycles, using a modified version of a publicly-available segmentation algorithm, discussed in the article by David Springer et al available from https://doi.org/10.13026/vnt9-kf93. For each segment the thirteen first MFCC's were computed. The resulting MFCC matrix was then resized to dimensions 13×200, forming the input data of the LSTM network. The predicted murmur-grade of a recording was obtained by taking the median activation-value across the six extracted segments.

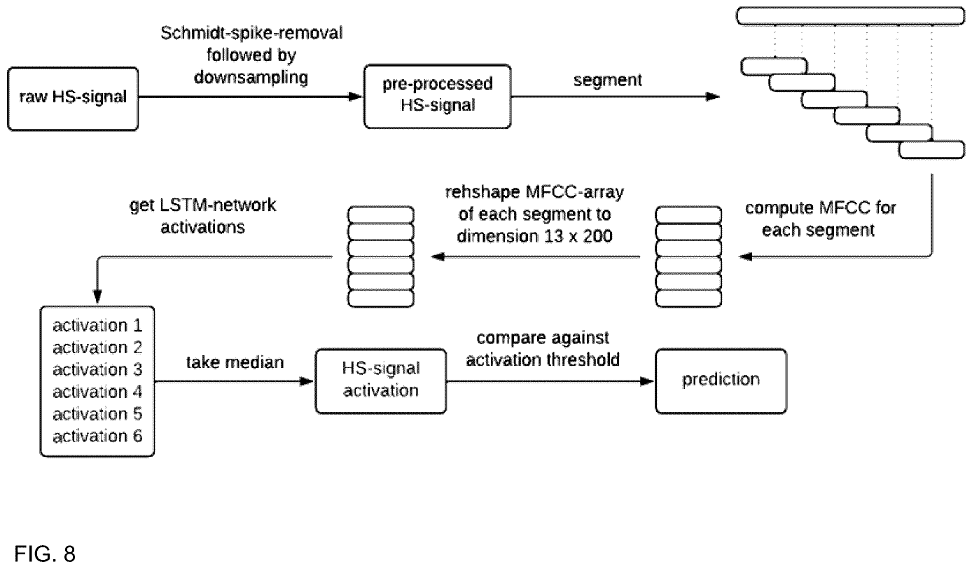

FIG. 8 provides a schematic overview of the steps between raw heart-sound (HS) signal input and the murmur-grade prediction.

A frequently occurring error that we observed was that Springer's segmentation algorithm failed to correctly estimate the heart-rate (HR) despite the audio being of high quality. We therefore made some small modifications to the publicly available code available at https://github.com/davidspringer/Springer-Segmentation-Code, and implemented a method that utilizes all four HS-recordings to produce a more robust HR-estimate.

### Overall Model Evaluation

Due to the small numbers of clinically relevant cases of VHD in the dataset, we opted to rely primarily on 8-fold cross validation (CV) to estimate test-performance. 1911 (90% of the data) observations were randomly selected to be used for cross-validation and model development, which we in the following will refer to as the development set. In each CV-split, the training and validation set consists of ˜1672 (87.5%) and ˜239 (12.5%) observations respectively. The CV-partitioning of the data is randomized in a way that ensures approximately equal numbers of VHD-cases and murmurs in each validation set. Partitioning is based on participant id-number, so that data from a participant never appears in both the training and validation set. Extensive use of CV for model selection can cause overestimation of actual test-performance, and several steps were therefore taken to minimize degree of overfitting to the development set.

Prior to performing any data-analysis, we set aside a holdout set containing data from 212 (˜10% of the data) participants which contains a total of 91 murmurs of grade 1 or higher, and 55 murmurs grade 2 or higher. All decisions related to the development of the murmur-detection model were done to improve prediction of murmur-grade, and not VHD-prediction.

After data analysis and model development had been performed on the development set, we retrained the murmur-detection algorithm on the whole development set and tested for overtraining on the holdout set.

### Statistical Analysis

Algorithm performance is measured primarily using Area-under-curve (AUC), sensitivity, and specificity. AUC was the preferred comparison metric in this study, as it is sensitive to both sensitivity and specificity, and doesn't require selection of a decision threshold. All confidence intervals (CI) and p-values disclosed here correspond to a significance level of 5%. AUC CI's are computed using standard methods under the assumption that the AUC-values of each CV-split are normal and independently distributed. We assume that CV-predictions are independent and identically distributed, and compute CI's for sensitivities and specificities using corresponding binomial distributions. Optimal decision thresholds were estimated on the training set of each CV-split by identifying the threshold that maximized sensitivity and specificity, subject to the condition that sensitivity should be at least 50%. Selection of optimal decision threshold was performed separately for each prediction target.

### Predicting AS Using Multiple Auscultation Positions

We hypothesized that more accurate prediction of mean AVPG could be achieved by aggregating murmur-grade predictions from all four recordings in a linear multivariate predictive model, rather than using only audio from a single predetermined position. In the following, these models are referred to as the multi-position and single-position models respectively.

As there were only 45 cases of at least mild AS in the development set, using AS classification accuracy for model selection would likely result in overfitting. Therefore, we based model selection on the ability to predict the AVPGₘₑₐₙ as a continuous variable. As model candidates for the regression model, we considered linear regression models that contain up to 2nd degree terms, and also considered interaction terms that account for absence of information due to noisy recordings, in which case weights were adjusted to increase the influence of lower priority positions which have usable audio. Model selection was performed by starting with a base model with one term for each murmur grade, and then terms associated with low p-values are stepwise added or eliminated in order to find the sub-model with the best Bayesian information criterion (BIC), which measures goodness of fit relative to model complexity. After the best model was been estimated, we added, in a similar manner, the interaction terms.

For testing the resulting models, we consider a total of eight different benchmark predictors, each of which are defined below according to the way that they summarize the predicted murmur-grades:
1. M₁=M_{A}: Aortic-only prediction model
2. M₂=M_{P}: Pulmonic-only prediction model
3. M₃=M_{T}: Tricuspic-only prediction model
4. M₄=M_{M}: Mitral-only prediction model
5. M_{maxAP}: maximum of Aortic and Pulmonic prediction
6. M_{sumAP}: sum of Aortic and Pulmonic prediction
7. Mₘₐₓ: max of all predictions
8. Mₛᵤₘ: sum of all predictions

Note that models 1, 2, 5 and 6 are intended to reflect standard guidelines for clinical practice, where typically the aortic position and sometimes the pulmonic position is referred to as the preferred position(s) to listen for AS murmurs and instances of high AVPGₘₑₐₙ. For each benchmark model, we form a prediction of the AVPGₘₑₐₙ from the corresponding murmur-grade summary variable Z by fitting a model of the form

$$AVPGmean = {β}_{0} + {β}_{1} \cdot Z + ϵ$$

We fit these models in order to compare the root-mean-squared-error (RMSE) and mean-absolute-error (MAE) of the benchmark methods against those of the proposed method, and thereby each models ability to predict AVPGₘₑₐₙ directly.

### Evaluation Metrics

The primary metrics we use to evaluate the models are
the RMSE between predicted and actual AVPGₘₑₐₙ
the MAE between predicated and actual AVPGₘₑₐₙ
the correlation between predicted and actual AVPGₘₑₐₙ

Since it is often of interest to predict exceedance of AVPGₘₑₐₙ above a specified threshold, we also consider the ability to predict cases where AVPGₘₑₐₙ≥u for different thresholds u. We assess this ability in two ways:
1. By treating the outputs of each calibrated model as activation values which we then use to compute the ROC area-under-the-curve (AUC). This gives us an overall impression of how good the model is at predicting exceedances above u without the need to specify a decision threshold. In the following we denote the AUC for predicting AVPGₘₑₐₙ≥u with AUCᵤ. As the selection of threshold would be arbitrary, we summarize the AUC metrics by averaging the AUCᵤ across u in the set {5, 6, 7, . . . , 25}, and refer to this metric as the average AUC.
2. We calculate the accuracy for predicting AVPGₘₑₐₙ≥u by defining a binary decision variable d which takes value 1 whenever predicted (AVPGmean)≥u. Again, to lose the dependence on threshold selection, metric the average accuracy across the thresholds u in {5, 10, 15, 20, 25}, and refer to this metric as the average accuracy.

The reason for including the second accuracy metric is to have an additional metric to reflect model interpretability, in addition to linear correlation. These metrics are considered since it is ultimately a human observer which will make a decision based on the algorithms numerical output, so we regard the interpretability of that output as an important aspect of model performance in itself.

### Statistical Tests

To test for statistical significance of performance differences we perform 8-fold cross validation (CV), and test for significance by performing paired t-tests (two-sided) on the estimates generated for each CV-split on the validation set. The results of these tests are reported as p-values.

### Performance Metrics

The table below shows comparison results for the AVPGₘₑₐₙ-calibrated model against the benchmark models in terms of four different performance metrics, with p-values (AVPGₘₑₐₙ-calibrated vs benchmark) for the paired t-test shown in the brackets. The results are obtained from 8-fold cross-validation. Average accuracy is defined as the accuracy of predicting AVPGₘₑₐₙ≥u averaged across thresholds u in the set {5, 10, 15, 20, 25}. RMSE=root-mean-square-error, MAE=mean-absolute-error.

| | Correlation | | RMSE | | MAE | | Avg. acc. | |
|---|---|---|---|---|---|---|---|---|
| AVPGₘₑₐₙ | 0.781 | | 2.62 | | 1.34 | | 0.954 | |
| Mₛᵤₘ | 0.717 | (0.005) | 2.7 | (0.3) | 1.47 | (0.005) | 0.947 | (0.02) |
| Mₘₐₓ | 0.614 | (0.001) | 3.1 | (0.05) | 1.63 | (0.001) | 0.936 | (0.001) |
| M_{sum, AP} | 0.675 | (0.000) | 2.94 | (0.070) | 1.53 | (0.002) | 0.945 | (0.006) |
| M_{max, AP} | 0.673 | (0.000) | 3.04 | (0.050) | 1.59 | (0.001) | 0.941 | (0.000) |
| M_{A} | 0.62 | (0.001) | 3.08 | (0.050) | 1.6 | (0.000) | 0.94 | (0.000) |
| M_{P} | 0.663 | (0.000) | 2.95 | (0.06) | 1.54 | (0.001) | 0.944 | (0.001) |
| M_{T} | 0.674 | (0.000) | 3.01 | (0.08) | 1.56 | (0.002) | 0.943 | (0.000) |
| M_{M} | 0.609 | (0.000) | 3.13 | (0.008) | 1.61 | (0.001) | 0.941 | (0.004) |

As can be seen from the table, the AVPGₘₑₐₙ-calibrated model significantly outperformed (p≤0.005) all the benchmark models in terms of linear correlation, with a linear correlation of 0.781; an increase of 0.064 compared to the second highest linear correlation score of 0.717 corresponding to Mₛᵤₘ. It also significantly outperforms all the benchmarks in terms of MAE (p≤0.005) as well as average accuracy (p≤0.02). The hardest benchmark to beat appears to be the summation model Mₛᵤₘ, which has only slightly higher RMSE and MAE.

The only metric for which the outperformance was not significant in all comparisons was the RMSE, despite the fact that the other measure of average prediction error, the MAE, was very significantly different in all comparisons. This apparent inconsistency is likely due to the RMSE being sensitive to extreme outliers, as it depends on the square of each error. As an example, consider a case where the AVPGₘₑₐₙ=50 and the predicted value is 20, then the contribution to the sum of squared errors is (50−20)²=900. Now compare this to contribution corresponding to prediction of a normal value, say 4.1 and a predicted value of 5.1; the contribution to the sum of squared errors would be just 1, despite both predictions being decent. Hence, the RMSE will have a very high dependence on a small portion of the validation data, resulting in high random variability, and a subsequent loss of statistical power in the results.

### AUC Comparison

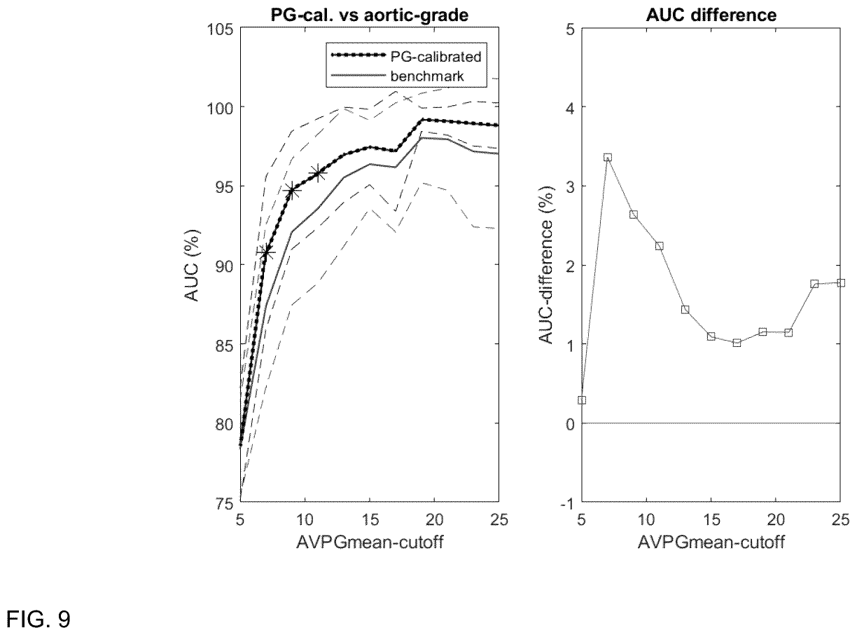

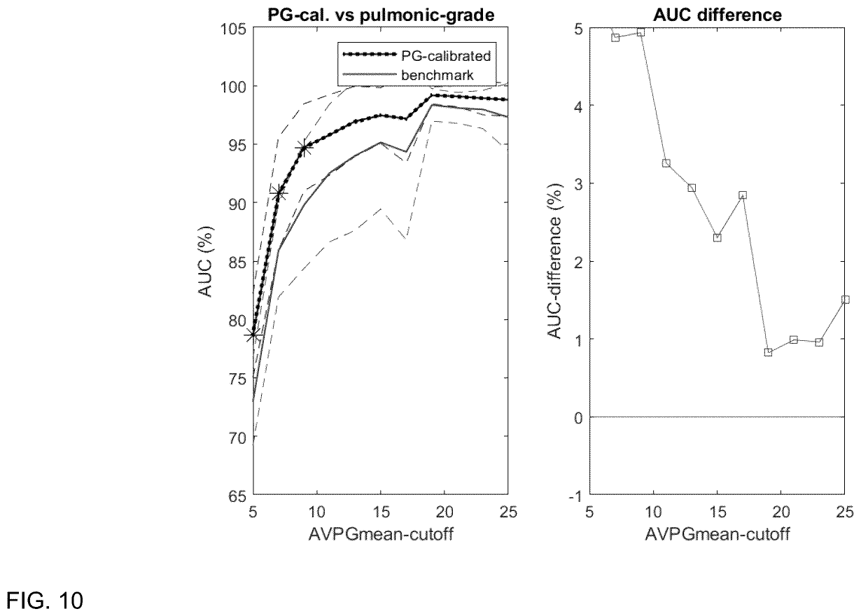

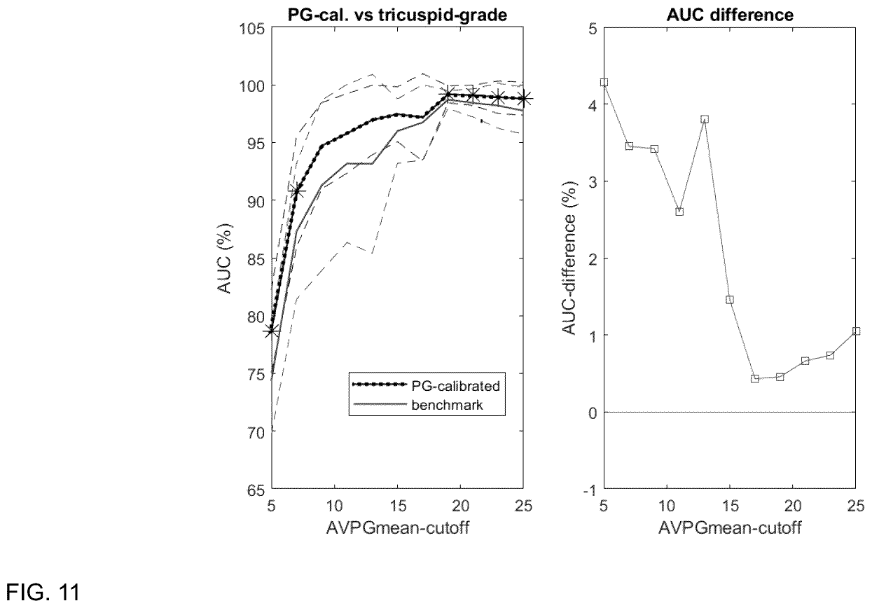

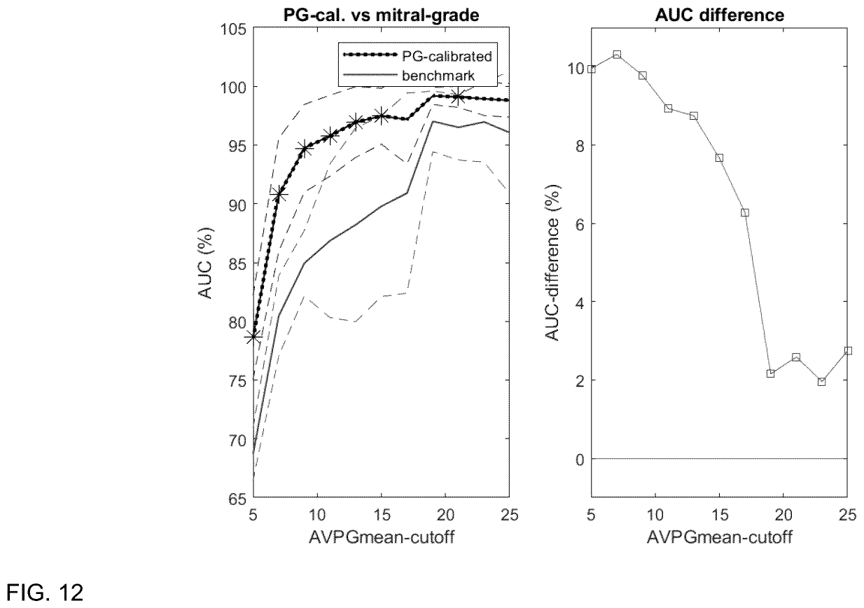

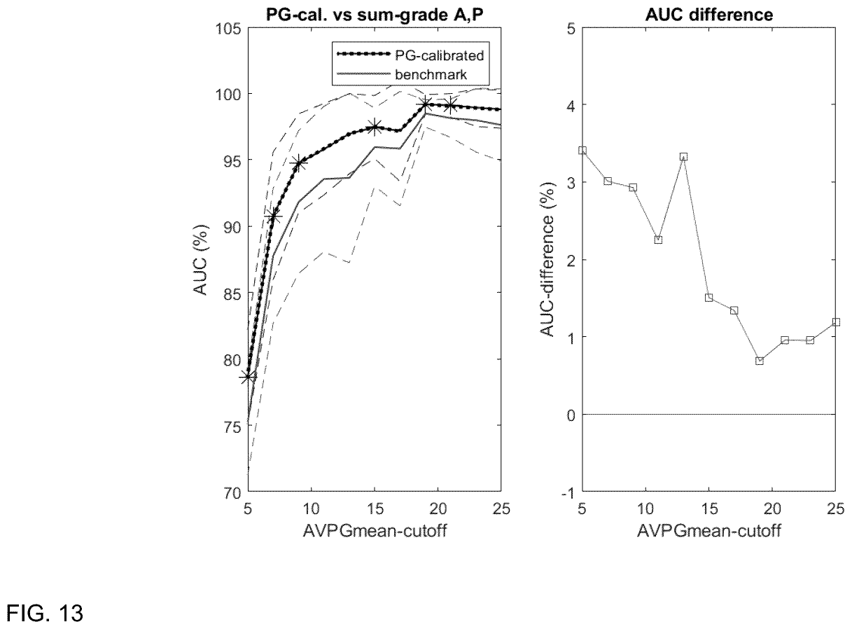

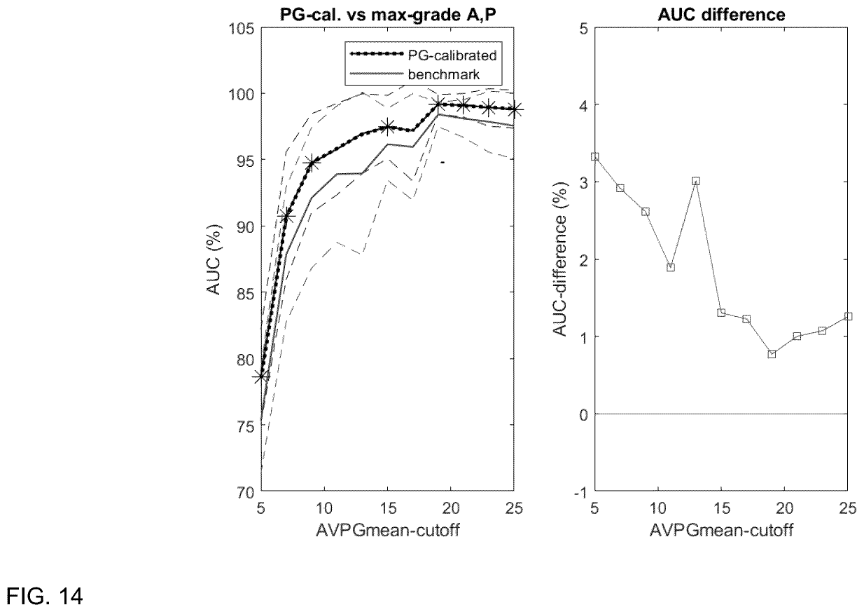

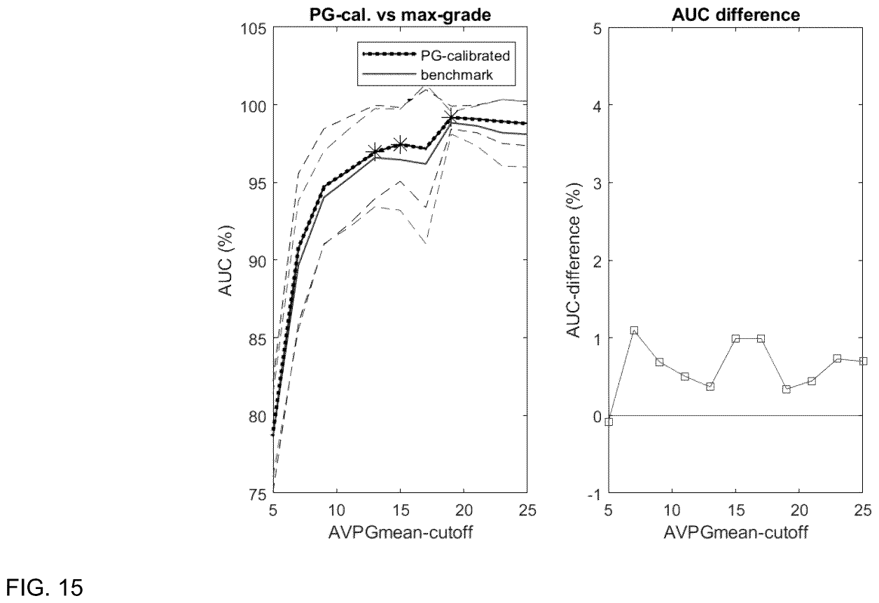

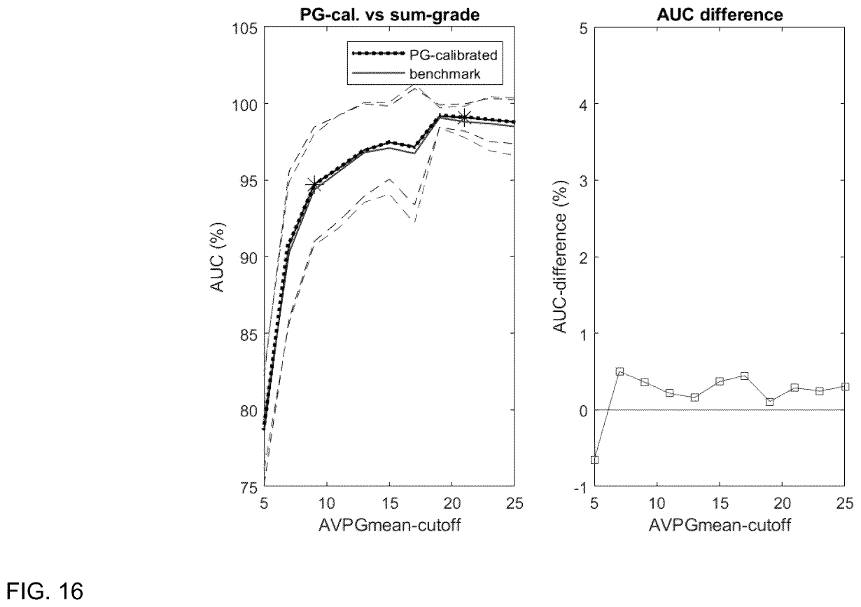

FIGS. 9-16 show, in the left panel of each figure, area under curve (AUC) as a function of cut-off threshold for the exceedance class, with each figure comparing the AVPGₘₑₐₙ-calibrated model against a different respective benchmark model. Significant differences are marked on the graph with asterisks (*). Each graph also shows upper and lower lines representing 95% confidence intervals, generated using bootstrapping, for the average pressure gradient (PG) model and for the respective benchmark model. The right panel of each figure shows the difference in AUC as a function of the cut-off threshold.

The table below shows which of FIGS. 6-19 corresponds to which benchmark model.

| Figure | Model |
|---|---|
| 9 | M₁ |
| 10 | M₂ |
| 11 | M₃ |
| 12 | M₄ |
| 13 | M_{sum, AP} |
| 14 | M_{max, AP} |
| 15 | Mₘₐₓ |
| 16 | Mₛᵤₘ |

In the FIGS. 9-16, the AUC for predicting {AVPGₘₑₐₙ≥u} has been computed across a range of thresholds in order to get a summary statistic with which to compare the models. The * indicate a difference corresponding to p<0.05. Results showing the significance of the performance difference in terms of the average AUC is shown in the following table, which gives p-values for paired t-tests for the AUC corresponding to the target AVERAGE (AUCᵤ|u in {5, 6, . . . , 25}).

| Model | p-value |
|---|---|
| Mₛᵤₘ | 0.3 |
| Mₘₐₓ | 0.006 |
| M_{max, AP} | 0.004 |
| M_{sum, AP} | 0.002 |
| M_{A} | 0.07 |
| M_{P} | 0.04 |
| M_{T} | 0.07 |
| M_{M} | 0.004 |

Overall, the AVPGₘₑₐₙ-calibrated model seems to outperform the single-position models, although significance is reached only for some of the thresholds. The summary metrics in this table shows that it reaches near-significant (p≤0.07) outperformance when compared against each single position model. We see that the difference with the Mₛᵤₘ is not significant (p=0.3). All the other p-values are significant or near significant (p≤0.07) however.

### Murmur Detection Performance

Combining predictions across all four auscultation positions, the murmur-detection algorithm predicted murmur-grade at least 2 with AUC of 0.969 (CI: 0.958-0.982), sensitivity 86.5% (CI: 83.2%-89.7%) and specificity 95.2% (CI: 94.7%-95.7%). When lowering the murmur-grade threshold to include grade 1 murmurs, the AUC decreased to 0.940 (CI: 0.931-0.949).

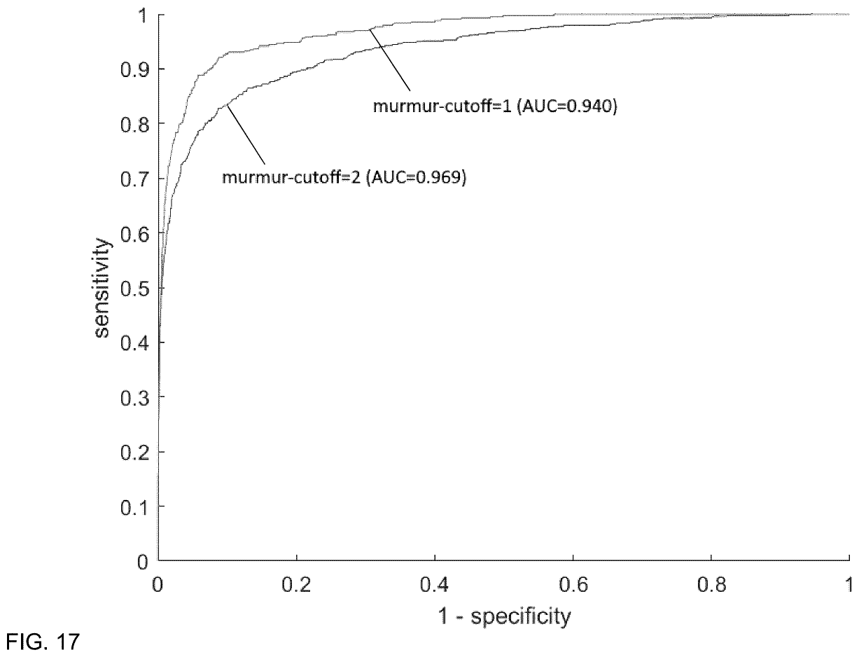

FIG. 17 shows receiving operator characteristics curves illustrating the algorithm's ability to detect murmur-grade of at least 1 (blue) and of at least 2 (red) respectively, and shows trade-off between sensitivity and specificity for different decision thresholds.

The holdout set contained a total of 55 murmurs grade at least 2, and the holdout-set AUC for prediction of murmurs was 0.982 when including murmur-grade 2 and higher (n=55), and 0.935 when including murmur-grade 1 and higher (n=91). As we observe no drop in performance on the holdout set, we conclude that there is no indication of overfitting for the murmur-detection algorithm.

In summary, the murmur-detection algorithm was highly accurate at predicting AS, especially when using the AVPGₘₑₐₙ calibrated model. An advantage to using the AVPGₘₑₐₙ calibrated model is increased robustness to noisy audio, as we observed no notable drop in performance when extending prediction to subsets that included noise in up to 3 positions.

A theoretical reason for preferring the multiposition model is that taking a weighted average may dampen the random variability in the predictions of each position. We have shown that mumur-grade predictions can be more efficiently utilized when taken in combination, rather than using a select subset for prediction following some predetermined preference.

Taking the average murmur grade from two clinicians annotating each recording in the training labels for the murmur grade prediction algorithm was also found to be particularly advantageous for improving the performance of the overall murmur-grading and regression model. Experiments were conducted to compare the performance of regression models trained using individual annotations (i.e. integer grade values) with the average of murmur-grade annotators of two clinicians as the input murmur-grade values (i.e. including non-integer values).

The performance of the regression model trained on the average of the annotations exceeded the performance of the regression models trained on individual annotations. The Applicant's tests showed that taking the average of the annotators' murmur evaluations produces a value that is more predictive of AS than using non-averaged evaluations from a single clinician.

It will be appreciated by those skilled in the art that the invention has been illustrated by describing one or more specific embodiments thereof, but is not limited to these embodiments; many variations and modifications are possible, within the scope of the accompanying claims.

1. A non-transitory computer-readable storage medium storing computer software for assessing health of a heart valve of a heart of a subject, the computer software comprising instructions, which, when executed on a processing system, cause the processing system to carry out a method comprising:
receiving a plurality of murmur-grade values, wherein each murmur-grade value is associated with a different respective auscultation position;
inputting the plurality of murmur-grade values to a first trained software model, implemented by the computer software;
operating the first trained software model to generate a value indicative of health of the heart valve, wherein the first trained software model uses murmur-grade values associated with two or more different auscultation positions when generating the value indicative of health of the heart valve; and
outputting the value indicative of health of the heart valve from the first trained software model.

2. The non-transitory computer-readable storage medium of claim 1, wherein the value indicative of health of the heart valve correlates with a pressure gradient across the heart valve.

3. The non-transitory computer-readable storage medium of claim 1, comprising instructions for receiving at least one of the murmur-grade values as a non-integer value.

4. The non-transitory computer-readable storage medium of claim 1, comprising instructions for receiving two, three or four murmur-grade values, each murmur-grade value being associated with a different respective one of an aortic, pulmonary, tricuspid or mitral auscultation point.

5. The non-transitory computer-readable storage medium of claim 1, wherein the first trained software model is a regression model.

6. The non-transitory computer-readable storage medium of claim 5, wherein the first trained software model is a multi-variate linear regression model.

7. The non-transitory computer-readable storage medium of claim 1, wherein the first trained software model has been trained using supervised learning on training data comprising training tuples, each training tuple comprising i) a plurality of murmur-grade values associated with a corresponding plurality of different auscultation positions for a respective heart, and ii) a ground-truth value indicative of the health of a valve of the respective heart.

8. The non-transitory computer-readable storage medium of claim 1, comprising instructions for:
receiving a plurality of sound recordings of the heart, wherein each sound recording is or has been captured by a microphone positioned at a different respective auscultation position; and
for each sound recording, inputting the sound recording to a second trained software model, implemented by the computer software, and operating the second trained software model to output a murmur-grade value associated with the respective auscultation position.

9. The non-transitory computer-readable storage medium of claim 8, wherein the second trained software model generates all of the murmur-grade values that are input to the first trained software model.

10. The non-transitory computer-readable storage medium of claim 8, wherein the second trained software model comprises a trained neural network.

11. The non-transitory computer-readable storage medium of claim 8, wherein the second trained software model is configured to process each sound recording without using information that identifies which auscultation position the sound recording was obtained from.

12. The non-transitory computer-readable storage medium of claim 8, wherein the second trained software model has been trained using supervised learning on training data comprising training tuples, each training tuple comprising i) a respective sound recording of a respective heart, and ii) a respective ground-truth murmur-grade value for the sound recording.

13. The non-transitory computer-readable storage medium of claim 12, wherein each ground-truth murmur-grade value has been determined by one or more clinicians listening to the respective sound recording and determining a murmur-grade value.

14. The non-transitory computer-readable storage medium of claim 1, comprising instructions for:
receiving a respective quality indicator value for each of the plurality of received murmur-grade values; and
inputting the quality indicator values to the first trained software model.

15. The non-transitory computer-readable storage medium of claim 14, wherein each quality indicator is a binary measure, and the method carried out by the processing system comprises the first trained software model using each binary measure to determine whether or not to use the respective murmur-grade value when generating the value indicative of the health of the heart valve.

16. The non-transitory computer-readable storage medium of claim 14, comprising instructions for operating a trained software model, implemented by the computer software, to generate the plurality of quality indicator values, wherein the first trained software model is configured to receive a sound recording of the heart as input and to output a murmur-grade value and a quality indicator value for the sound recording.

17. The non-transitory computer-readable storage medium of claim 1, wherein the heart valve is an aortic valve and wherein the value indicative of health of the heart valve is indicative of the presence of aortic stenosis.

18. A computer system for assessing the health of a heart valve of a heart of a subject, wherein the computer system comprises a processing system and a memory storing computer software for execution by the processing system, and is configured to:
receive a plurality of murmur-grade values, wherein each murmur-grade value is associated with a different respective auscultation position;
input the plurality of murmur-grade values to a first trained software model, implemented by the computer software;
operate the first trained software model to generate a value indicative of health of the heart valve, wherein the first trained software model uses murmur-grade values associated with two or more different auscultation positions when generating the value indicative of health of the heart valve; and
output the value indicative of health of the heart valve from the first trained software model.

19. A method for assessing health of a heart valve of a heart of a subject, wherein the method is performed by a processing system and comprises:
receiving a plurality of murmur-grade values, wherein each murmur-grade value is associated with a different respective auscultation position;
inputting the plurality of murmur-grade values to a first trained software model;
operating the first trained software model to generate a value indicative of health of the heart valve, wherein the first trained software model uses murmur-grade values associated with two or more different auscultation positions when generating the value indicative of health of the heart valve; and
outputting the value indicative of health of the heart valve from the first trained software model.

20. The method of claim 19, wherein the heart valve is an aortic valve and wherein the value indicative of health of the heart valve is indicative of the presence of aortic stenosis.
